(19) United States
(12) Patent Application Publication
(10) Pub. No.: US 2006/0263440 A1
(43) Pub. Date: Nov. 23, 2006

(54) Acne treatment

(76) Inventor: Jackie J. Cecil, Valdosta, GA (US)

Correspondence Address: John P. Sinnott, 1007 N. Patterson Street, Valdosta, GA, 31601, US

(21) Appl. No.: 11/132,935

(22) Filed: May 19, 2005

(51) Int. Cl. A61M 31/00; A61K 36/13; A61K 35/64; A61K 33/14; A61K 33/00

(52) U.S. Cl. 424/539; 424/600; 424/680; 424/770; 604/500

(57) ABSTRACT

An effective treatment for removing acne blemishes with non-prescription substances is described. The afflicted area first is washed with very warm water and an emulsifier. Hot towels are applied to the blemishes and the towels are removed. The skin is left moist, followed by application of a hot cloth soaked in saline water. Pairs of steaming hot towels are placed over the saline water soaked cloth until the subject feels that each of the pairs of towels are no longer hot. The afflicted area then is washed with a castile soap and residual brown spots on the skin are post treated with a topical application of turpentine or honey.

### CROSS-REFERENCES TO RELATED APPLICATIONS

None

### STATEMENT REGARDING FEDERALLY SPONSORED RESEARCH OR DEVELOPMENT

None

### REFERENCE TO “SEQUENCE LISTING”

None

### BACKGROUND OF THE INVENTION

This invention relates to methods for treating acne and, more particularly, to a procedure for opening the pores by applying several hot, steaming towels to the afflicted area followed by application of a hot towel soaked with hot saline water and washing the affected area with castile soap to remove the blemishes, and the like.

It is curious that in a time of great advances in medicine, a cure for the common affliction of acne, or at least a truly effective method for eliminating the unsightly red pimples that characterize this condition continues to escape effective treatment. This is all the more unfortunate when the damaging effect this condition can impose on the life of a young person is taken into consideration.

Through the years a number of techniques have been advanced to remove acne blemishes from an afflicted person's skin. For example, application of silver citrate and a soap to the afflicted area has been proposed for acne blemish removal. To a greater or lesser extent these proposals have failed to fully satisfy the need for a simple and effective non-prescription method to eliminate what in all too many instances is a serious facial disfigurement.

### BRIEF SUMMARY OF THE INVENTION

Through the practice of invention, the red pustules that characterize acne can be reduced to a degree in which the pustules are eliminated and the only visible residue are a few circular, brown areas, somewhat darker in color than ordinary freckles, in those places in which the acne pimples were concentrated.

Illustratively, to remove from a person's skin the red pimples that characterize acne, the subject's skin is washed with warm to hot water and a cleansing agent that is characterized by a high concentration of emulsifiers. Hot steaming towels then are applied to the afflicted area at least three times. The skin, left moist, has a hot salt water saturated towel placed on the afflicted area with two steaming towels placed over the salt water soaked towel. This salt water treatment step is repeated once more, the towels being removed when the subject feels that the steaming towels are no longer hot. A castile soap then is used to wash the treated skin.

After treatment, the skin is slightly flushed for several hours. By the third day after treatment, the red pustules are gone, leaving only a few brown, freckle-like areas in those places that were most seriously affected by the pustules. These brown blemishes then are post treated through a local, topical application of turpentine or honey.

Thus, the practice of the invention provides an improved method for removing the blemishes that characterize an acne skin condition.

### DETAILED DESCRIPTION OF INVENTION

In accordance with the invention, acne pustules are eliminated from an afflicted area of skin through the following steps:
a. The afflicted area is washed with a warm to hot (e.g. a temperature of 110° F. or somewhat less) water and a soap that has a high emulsifier content. For example, it has been found that Ultra Palmolive® Concentrated Dish Liquid Antibacterial Hand Soap marketed by the Colgate-Palmolive Company of New York, N.Y.¹ is satisfactory for the purpose of the invention. With respect to this step in the process, it is believed that the warm to hot water opens the pores in the skin, reduces the viscosity of the matter in the pustules and conditions the skin to receive steamed towels that are later to be applied. The Palmolive ® soap, moreover, being high in emulsifiers is thought to encapsulate and remove the matter from the pustules through the opened pores.
   The Ultra Palmolive® composition's active ingredient is triclofan. The inactive ingredients are: water, ammonium laureth sulfate, lauryl polyglucose, magnesium and sodium sulfonates, SD3A alcohol, lauramide myristamide, sodium xylene sulfonate, magnesium sulfate, sodium chloride, fragrance, trisodium hedta, quaternium 15, sodium bicarbonate, DC orange 4, and methenamine HC_{L}.
b. Hot steaming towels then are applied to the afflicted area at least three times and, afterward, the afflicted area is patted dry.
c. One tablespoon of common table salt is dissolved in one measuring cup of very hot water having a temperature of not more than 120° F. The salt is completely dissolved in the water and a facecloth is immersed and soaked in the salt water. Sea salt with naturally occurring substances may have antibacterial characteristics that are superior to ordinary table salt and thus, sea salt water may be preferable for use in this step of the treatment process. The salt kills, it is believed, through osmosis.
d. A towel is folded lengthwise and then across. One side of the towel is thoroughly saturated with ordinary tap water and then folded into thirds in order to keep the wetted side of the towel on the inside of the folds. The towel is heated, preferably in a microwave oven, for one minute in order to heat the water in the wetted interior until it steams.
e. With the subject's head slightly inclined to prevent inhalation of water from the towel (when treating facial acne), the hot salt water soaked facecloth is placed over the affected area, the saline water soaked facecloth then being covered by the unfolded steaming towel. Preferably, a second steaming towel can be placed over the first towel to maintain the facecloth's heat for a longer period of time. Care must be taken, however, to make sure that these hot applications are sufficiently cool to avoid burning the skin that is undergoing treatment. With respect to this step in the treatment, it is thought that the hot applications help to further open the pores and gravity promotes flow of the saline water from the facecloth into the pores. The heat, it seems, also promotes an increase in capillary blood flow that not only increases the supply of white and red blood cells to advance the healing process, but also removes waste products from the afflicted area. The effect of the heat applied to the subject's skin also might destroy at least some of the bacteria that are related to the acne condition. As a general matter, heat may aid in the process in several ways. It may denature proteins, cause membrane damage and also may cause enzymatic cleavage of DNA. Moist heat, moreover, sterilizes at a lower temperature than dry heat because water serves to disrupt noncovalent bonds (hydrogen bonds) which hold protein chains together in their secondary and tertiary structures.
f. The steaming towel is replaced at least one more time, this replacement being repeated when the subject feels that the applied towel is no longer hot or has a noticeably elevated temperature. In this instance, the towel may actually be very warm, but the subject has at this point in the treatment become so conditioned to heat that the sensation of heat fails to register with the patient as being significant. The process temperatures need to be continually increased to and maintained at the desired levels to cause morbidity of proionibacterium acnes, dilation of the body's own vascular structures to bring in the body's own defensive mechanisms, remove waste through the dilated structures, and allow the salt to enter deep into the affected pilosebaceous unit. The salt will, but is not limited to, deprive the bacterium of water needed to survive, and the sodium that enters the bacterium's membrane can not be excreted (it is a one-way trap for sodium, much like a diode is in electricity), but more water has to be brought into the bacteria to dilute the absorbed sodium until the bacteria actually ruptures.
g. After the last steaming towel is felt to be cool by the subject, the steaming towels and the underlying saline water soaked facecloth are removed.
h. The afflicted area then is further treated by being washed with a castile soap. For the purpose of the invention, the soap marketed as “Dr. Bronner's Magic Soaps 18-in-1 Hemp Peppermint Pure-Castile Soap” has been found to be particularly efficacious. The Dr. Bronner's soap that is used in the invention is a compound of water, saponified coconut-hemp-olive oils (with retained glycerin), olive fatty acids, peppermint oil (boosted with menthol), vitamin E, and citric acid.
i. The treated area is rinsed and dried, having on visual inspection a rosy appearance that will disappear in about twelve hours.

Within three days after treatment, the red pustules will be gone from the afflicted area, leaving only a few circular, dark brown freckle-like areas in those portions of the skin that were most seriously afflicted. To post treat these residual marks, a cotton applicator, soaked in either turpentine or in honey, is touched to the dark areas.

As a result, a better and more efficacious treatment for acne now is possible in which treatment of an afflicted person can be accomplished without resort to expensive prescription pharmaceuticals and the like. Throughout the process, a primary goal is to increase the temperature in the dermis layer where the “pilosebaceous unit” and the acnes' effects are present. The believed causative agent is propionibacterium acnes; it best survives in the temperature range of the human body, plus or minus a few degrees. It is also an anaerobic type bacterium which means it best lives in an environment lacking in oxygen, i.e., the plugged, or occluded, pilosebaceous unit. That is why the basis of treatment begins with unplugging the follicular opening and allowing air into the unit and beginning to raise the temperature of the dermis at the same time.

What is claimed is:

1. A method for treating skin of a person afflicted with acne pustules, comprising the steps of washing the pustules with very warm water and an emulsifying agent, applying a plurality of hot towels over the pustules, applying hot saline water to the pustules, and washing the pustules with a castile soap.

2. An acne treatment method according to claim 1 further comprising the step of post treating the skin with a substance selected from the group comprising honey or turpentine.

3. An acne treatment method according to claim 1 further comprising the steps of soaking a cloth in hot saline water and placing said saline water soaked cloth on the pustules.

4. An acne treatment method according to claim 1 wherein said hot saline water application comprises the step of dissolving sea salt in said hot water.

5. An acne treatment according to claim 3 further comprises the steps of applying a first steaming towel to said hot saline water soaked cloth, applying another steaming towel to said first towel, and removing said towels when the person does not have a sensation of elevated temperature from said towels.

6. An acne treatment according to claim 5 comprising the steps of applying another set of two steaming towels to said hot saline water soaked cloth and removing said set of towels when the sensation of heat fails to register with the person as being significant.

7. An acne treatment according to claim 1 wherein said very warm water for washing the pustules is a temperature of not more than 110° F.

8. An acne treatment according to claim 1 wherein said hot saline water has a temperature of not more than 120° F.
